(19) United States
(12) Patent Application Publication
(10) Pub. No.: US 2020/0305683 A1
(43) Pub. Date: Oct. 1, 2020

(54) METHOD FOR MANUFACTURING AN ENDOSCOPE

(71) Applicant: OLYMPUS Winter and Ibe GmbH, Hamburg (DE)

(72) Inventors: Uwe SCHOELER, Hoisdorf (DE); Nils TORKUHL, Vollstedt (DE)

(73) Assignee: OLYMPUS Winter and Ibe GmbH, Hamburg (DE)

(21) Appl. No.: 16/832,687

(22) Filed: Mar. 27, 2020

(30) Foreign Application Priority Data
Mar. 28, 2019 (DE) 10 2019 108 117.3

(51) Int. Cl. A61B 1/00; B23K 1/00; B23K 1/002

(52) U.S. Cl. CPC A61B 1/0011; A61B 1/00096; B23K 2101/20; B23K 1/002; B23K 1/0008

(57) ABSTRACT

A method for the manufacturing an endoscope, the method including: providing a window having a lateral peripheral surface and a flat surface, the window is provided with a metal coating on a lateral peripheral surface and/or an edge region of the flat surface, and the flat surface of the window is provided with a solder preform, inserting the window provided with the solder preform into a window seat of an endoscope shaft of the endoscope, where the flat surface of the window one of faces an interior space of the endoscope shaft or faces away from the interior space of the endoscope shaft, and subsequent to the inserting, heating the solder preform with a heating device such that solder material of the solder preform is distributed between the window and the window seat and, after the solder material cools, solder is arranged between the window and the window seat.

### CROSS-REFERENCE TO RELATED APPLICATION

The present application claims the benefit to DE 10 2019 108 117.3 filed on Mar. 28, 2019, the entire contents of which is incorporated herein by reference.

### BACKGROUND

### Field

The present disclosure relates to a method for manufacturing an endoscope as well as the use of a window and a solder preform for manufacturing an endoscope having an endoscope shaft.

### Prior Art

Video endoscopes normally have a window on their proximal end in order to be able to look at objects to be observed on the distal end or near the distal end of the endoscope. Inside the video endoscope in the area of the window, i.e. on the proximal end, a camera can be provided, for example, which can take pictures through a corresponding window, which is provided on the distal end of the endoscope. Alternatively, optical fibers and, if applicable, optics can be provided in order to direct pictures from the distal end of the endoscope to the proximal end.

With endoscopes used in medical technology, it is necessary that the endoscopes can be sufficiently cleaned and sterilized. For this, they must be autoclavable, dishwasher-safe and also suitable for being stable in an aggressive chemical environment. A window in a, for example, rigid endoscope must therefore be installed in a hermetically sealing manner. For this, it is known to metallize the side walls of the window that border the tube material after installation of the window into the tube in a multi-layered process in order to then subsequently solder them. This is relatively complex. Moreover, it is required to use a solder, which will not be corroded in particular when using corrosive chemicals to clean the endoscope.

Moreover, at the proximal end of the endoscope, there is for example an eyepiece with an ocular, that is, an optical assembly, from which the light entering the distal tip of the endoscope exits. Such an ocular provided in the hermetic chamber can be used to directly observe the surgical field with the naked eye. Frequently, a camera head is connected to the ocular so that the surgical field can be observed on a monitor, or the captured image data can be supplied to a connected image processing system. Such an endoscope is known for example from EP 0 501 088 A1.

The optical assemblies in an interior space of the endoscope, for example in its shaft, are separated from each other from an exterior space that surrounds the endoscope by an ocular window.

With rigid endoscopes or videoscopes, the optical systems are hermetically sealed at the distal end and at the proximal end with a sapphire disk that is inserted in a distal fiber tube tip, or respectively in a proximal ocular window frame. On the distal side, the joining process is accomplished by manual soldering, whereas on the proximal side, the sapphire disk is glued in the ocular window frame and secured with a union nut.

### SUMMARY

An object is to enable easy and reliable insertion of a window into an end, such as into a distal or proximal end of the endoscope, whereby an autoclavable as well as chemically stable endoscope can be produced.

Such object can be achieved by a method for manufacturing an endoscope comprising:

providing a window having one lateral peripheral surface and two flat surfaces, wherein the window is provided with a metal coating on one or more of a lateral peripheral surface and in an edge region of a flat surface of the window, and wherein a flat surface of the window, which can be provided with the metal coating, is provided with a solder preform,

inserting the window provided with the solder preform into a window seat of an endoscope shaft of the endoscope, wherein the flat surface of the window can be provided with the solder preform one of faces the interior space of the endoscope shaft and faces away from the interior space of the endoscope shaft,

heating, the solder preform either at several points, or, simultaneously, heating the entire solder preform with a heating device so that the flowable solder material of the solder preform is distributed between the window and the window seat and, after the solder material cools, solder is arranged between the window and the window seat.

By providing a solder preform that is arranged between the flat surface of the window inserted into the window frame and the interior space of the endoscope shaft, or between the flat surface of the window and the exterior space of the endoscope, easy soldering of the window with the window seat can be achieved. In so doing, the solder preform can be heated partially or completely by means of an inductive heating device or a laser, whereby the solder material of the solder preform becomes flowable and thereby flows into the gap between the window, or respectively the inserted window disk and the inner wall as well as the contact surface of the window seat for the window, whereby a hermetically and chemically stable solder layer is formed between the window and the window seat.

The lateral peripheral surface of the window and the flat surface of the window can each be provided with a metal coating in one embodiment. Moreover, the inner wall of the window seat facing the lateral peripheral surface of the window, and/or the contact surface of the window seat facing the flat surface of the window can also be provided with a metal coating. The metal coatings for the peripheral surface, and/or the flat surface of the window, and/or the metal coatings for the inner wall and the contact surface of the window seat can each have gold, or can be produced from gold.

The metal coating applied to the flat surface of the window can be annular and formed on the edge of the window, whereby the metal coating on the flat surface surrounds an inner region for passage of light through the window.

In this case, the window can be fixed in the window seat, or respectively window frame without adhesive, i.e., adhesive-free, which simplifies soldering the window in the window seat.

A window can be provided that has one lateral peripheral surface and two parallel flat surfaces, wherein the window is provided with a metal coating on the lateral peripheral surface and/or in the edge region of exclusively one flat surface of the window, and wherein a solder preform is applied onto the flat surface of the window, which can be provided with the metal coating. The window is inserted into a window seat of an endoscope shaft of the endoscope, wherein the flat surface of the window, which can be provided with the metal coating, and the solder preform face the interior space of the endoscope shaft, or face away from the interior space of the endoscope shaft.

An endoscope, such as a rigid endoscope, video endoscope or videoscope with a lens system can be manufactured, wherein at least one window is soldered in a window seat of the endoscope, or video endoscope, or videoscope.

A window, or window disk can be soldered to a distal fiber tube tip, or to a proximal ocular frame by means of the method.

An effect of completely and simultaneously heating the solder preform achieves a self-centering of the window within the window seat after the solder material cools.

Moreover, a void-free, or respectively nearly void-free soldering in the gap between the window seat and the window can be achieved.

According to the method, the metal coating of the window can comprise of gold or an outer layer of gold.

Moreover, the window seat can have surfaces which face the lateral peripheral surface of the window and the flat surface of the window, wherein at least one surface, or both surfaces, can have a gold coating or an outer gold layer. In this case, the surfaces of the window frame are a peripheral inner wall that faces the lateral peripheral surface of the window, and a contact surface for the window that faces the flat surface of the window. In one embodiment, the solder preform can be arranged between the flat surface of the window provided with a metal coating at the edge of the window and the annular contact surface of the window seat for the flat surface of the window.

In this regard, the solder preform can be configured in an annular or disk-shape.

The solder preform can be made of gold and tin. The solder preform can have a mixture of 80% gold and 20% tin.

In order to heat the solder preform, the heating device can be configured as an inductive heating device or as a laser device. By means of the inductive heating device or the laser device, such as a diode laser, the solder preform can be heated simultaneously and completely, whereby the window is soldered in the window seat of the endoscope shaft provided therefor.

The window can be designed cylindrical and/or as a, disk-shaped, flat glass.

The window can be produced from sapphire glass.

The window seat can be produced from steel or plastic, such as PEEK (polyetheretherketone). The window can accordingly be inserted in a window seat comprising steel, such as at a fiber tube tip. In another embodiment, the window can be soldered in an ocular frame consisting of plastic at the proximal end of the endoscope shaft.

The window seat can be formed at the distal end of the endoscope shaft, such as at a distal fiber tube tip of a fiber tube, or at the proximal end of the endoscope shaft, such as in a window seat of an ocular device, or respectively an ocular.

The metal coating can be formed on the flat surface of the window in a ring-shape and/or can have a ring width between 50 μm and 1000 μm, such as between 100 μm and 500 μm.

Furthermore, the object can be achieved by using a window and a solder preform for manufacturing an endoscope having an endoscope shaft, such as a rigid endoscope or video endoscope, wherein the window can have one lateral peripheral surface and two flat surfaces, wherein the window can be provided with one of a metal coating on the lateral peripheral surface and in the edge region of a flat surface of the window, and wherein a flat surface of the window, which can be provided with the metal coating, is provided with a solder preform, wherein the window is inserted in a window seat of the endoscope shaft, wherein the flat surface of the window provided with the solder preform faces one of the interior space of the endoscope shaft and faces away from the interior space of the endoscope shaft.

The solder preform can be heated at several points, or the entire solder preform can be simultaneously heated with a heating device, so that the flowable solder material of the solder preform is distributed between the window and the window seat and, after the solder material cools, solder is arranged between the window and the window seat.

Furthermore, according to one embodiment, the metal coating of the window can comprise gold or have an outer layer of gold.

The solder preform can be configured in an annular or disk-shape.

The solder preform can comprise gold and tin.

The window can be configured to be one or more of cylindrical and as flat glass.

The window can be produced from sapphire glass.

Furthermore, the metal coating can be formed on the flat surface of the window in a ring-shape and can have a ring width between 50 μm and 1000 μm, such as between 100 μm and 500 μm.

### BRIEF DESCRIPTION OF THE DRAWINGS

Further features will become apparent from the description of the embodiments together with the claims and the attached drawings. Embodiments can fulfill individual features or a combination of several features.

The embodiments are described below, without restricting the general idea of the invention, using exemplary embodiments with reference to the drawings, express reference being made to the drawings with regard to all details that are not explained in greater detail in the text. In the following:

FIG. 1 illustrates a schematic and simplified side view of a surgical instrument,

FIG. 2 illustrates a detailed view of an ocular device, in a schematic and simplified longitudinal section,

FIG. 3a, 3b illustrate partial schematic views of an arrangement of a window disk in a fiber tube tip of an endoscope before being soldered (FIG. 3a) and in a soldered state (FIG. 3b),

FIG. 4 schematically illustrates a view of an ocular device of a surgical instrument in a simplified longitudinal section.

In the drawings, the same or similar elements and/or parts are provided with the same reference numbers in order to prevent the item from needing to be reintroduced.

### DETAILED DESCRIPTION

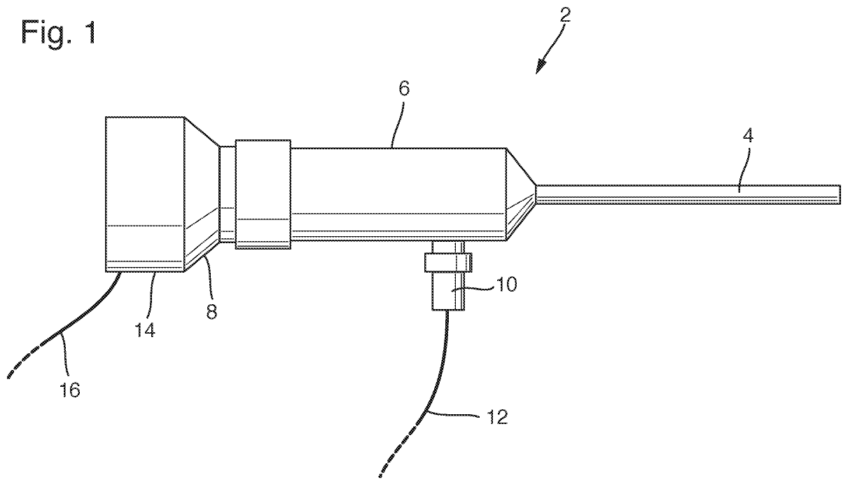

FIG. 1 shows a schematic and simplified side view of a surgical instrument 2 such as an endoscope. At its distal end, it comprises a tubular endoscope shaft 4 with a lens system that makes it possible to observe a surgical or investigated region lying distally in front of the free end of the endoscope shaft 4. The endoscope shaft 4 terminates in a housing 6 that has an eyepiece 8 at the proximal end. The housing 6 is for handling the surgical instrument 2. On the side of the housing 6 is a light source 10 such as an LED light source. This is connected by a connecting cable 12 to a suitable power supply.

A schematically shown camera head 14 with an ocular adapter (not shown) is arranged on the eyepiece 8. The camera head 14 detects the light exiting the ocular of the surgical instrument 2 with its own lens system, and images it on an optical surface sensor such as a CCD or CMOS chip. The camera head 14 is supplied with power by means of a connection 16. Furthermore, it is possible to send image signals by the connection 16 from the surface sensor of the camera head 14 to an external evaluation unit and transmit control signals to the camera head 14.

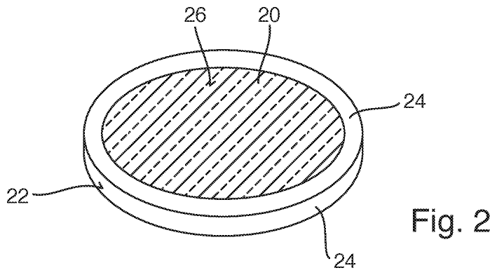

FIG. 2 schematically shows a perspective view of a window disk 20 that is inserted as a window, such as in a window opening, or respectively window seat at a distal fiber tube tip of a fiber tube 30 of an endoscope, or in a proximal window frame (window seat) of an ocular device. The window disk 20 in this case is configured cylindrically, or respectively cylinder-shaped with a cylindrical peripheral surface 22 and two flat surfaces 26, and has a gold coating 24 on its cylindrical peripheral surface 22. Given the perspective diagram, only one flat surface 26, i.e., the upper side, of the window disk 20 can be seen.

The window disk 20 can be configured as a flat glass and have an annular gold coating 24 in the edge region on the upper, or respectively visible flat surface 26. Only one of the two flat surfaces 26 of the window disk 20 has the annular gold coating 24 on the outer edge of the flat surface 26.

The window disk 20 can be produced from sapphire glass, wherein the window disk 20 is transparent, or respectively impermeable to visible light in the uncoated inner region that is framed, or respectively surrounded by the gold coating 24.

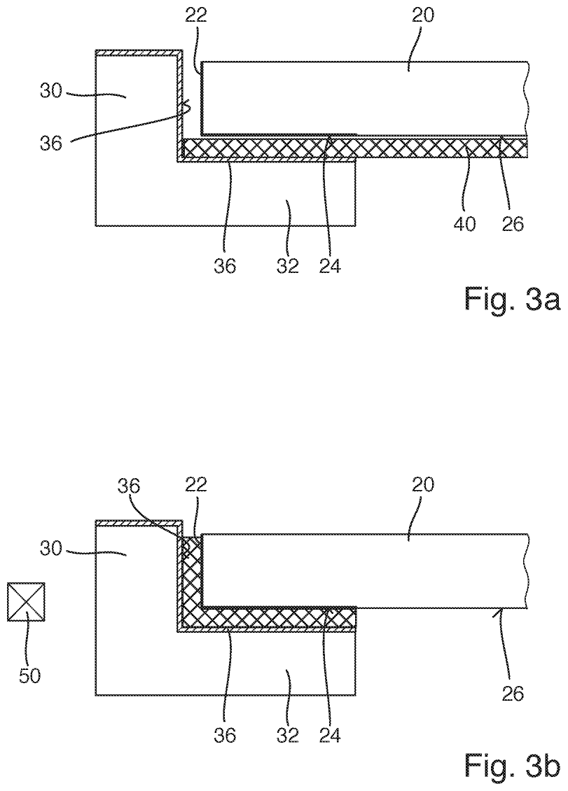

The window disk 20 is inserted into a corresponding window seat of a fiber tube tip (see FIG. 3a, 3b) and is bonded to the fiber tube by being soldered at the fiber tube tip so that the window disk 20 is soldered in the window frame of the fiber tube tip.

The coatings on the peripheral surface 22 and on the flat surface 26 of the window disk 20 can also have several layers, such as metal layers, wherein the outer layer of the multiple layers can be gold or contain gold.

The annular gold coating 24 on the top side, or respectively the flat surface 26 of the window disk 20 can have a width between 100 μm to 500 μm in this case. The width of the ring of the gold coating 24 on the flat surface 26 can be approximately 300 μm.

FIG. 3a schematically shows, in a section, a partial arrangement of the window disk 20 on a fiber tube tip of a fiber tube 30 at the distal end in a cross-section. In FIGS. 3a and 3b, only a portion of the window disk 20 and fiber tube 30 are illustrated. The same arrangement mirrored over a longitudinal center line of the window seat is not shown for simplicity. In the interior of the endoscope shaft 4 (see FIG. 1), the fiber tube tip is arranged on the distal end of the endoscope shaft 4. Optical components of the endoscope (not shown) are located in the interior of the fiber tube 30, typically at the distal end of the endoscope shaft 4.

At the distal end of the fiber tube 30, said tube is provided on the inside with a ring body 32 so that a contact surface for the edge region of the window disk 20 is formed on the inside of the ring body 32. FIG. 3a schematically shows the arrangement of the window disk 20 at the fiber tube tip of the fiber tube 30 before soldering in the window disk 20.

The fiber tube 30 is provided with a gold coating 36 on the inside on the contact surfaces facing the peripheral surface 22 of the window disk 20. Furthermore, the contact surface of the ring body 32 that faces the flat surface 26 of the window disk 20 is also provided with the gold coating 36. To solder in the window disk 20 in the frame, or respectively the window frame of the fiber tube tip, a solder preform 40 is arranged between the bottom side, or respectively the flat surface 26 of the window disk 20 which is provided with the gold coating 24 and the contact surface of the ring body 32 which is formed with the gold coating 36. The solder preform 40 in this case can be configured in an annular or disk-shape and is arranged between the window disk 20 and the ring body 32. The solder preform 40 can comprise gold and tin, such as 80% gold and 20% tin.

In order to solder the window disk 20 into the frame of the fiber tube tip, the solder preform 40 is heated by means of a schematically illustrated inductive heating device 50, whereby the solder material becomes flowable, and the solder material of the solder preform 40, due to capillary forces, therefore flows between the window disk 20 and the edge region between the window disk 20 and the fiber tube 30 in the gap between the fiber tube 30 and the window disk 20, whereby the edge region, or respectively the gap between the window 20 and the fiber tube 30, is filled with solder material, and the window disk 20 is thereby soldered into the fiber tube tip. This is shown in FIG. 3b in a partial schematic diagram.

FIG. 3b also schematically shows an inductive heating device 50 that surrounds the fiber tube tip for heating the solder preform 40. By energizing the inductive heating device 50 arranged outside of the fiber tube 30, the solder preform 40 is heated.

Simultaneously heating the complete solder preform 40 by means of the inductive heating device 50 allows the window disk 20 to be arranged and soldered in a self-centering manner in the opening in the fiber tube 30 due to the capillary forces between the window disk 20 and the fiber tube tip.

The solder preform 40 is designed disk-shaped or annular. The fiber tube 30 can be produced from metal.

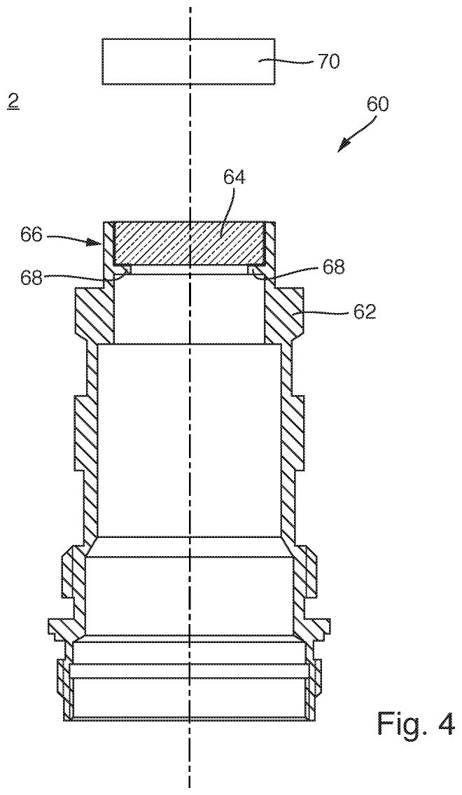

FIG. 4 schematically shows an ocular device 60 for a surgical instrument 2 in a schematic and simplified longitudinal section. The ocular device 60 is provided at the proximal end of a surgical instrument 2, such as an endoscope. The ocular device 60 in this case has an ocular window frame 62 in which an optical assembly is accommodated. The optical assembly is in this case one or more lenses or lens groups, prisms, filters, etc. that form an optical system.

FIG. 4 schematically shows the optical axis of the optical system in a dot-dashed line. The ocular device 60 is located in an interior space of the surgical instrument 2 such as an interior space enclosed by the housing 6 of the endoscope depicted in FIG. 1. The ocular window frame 62 can form the separation between the hermetic inner space and the non-hermetic outer space.

The ocular window frame 62 has an ocular window 64 at the light exit side. The ocular window 64 in this case is accommodated in a window frame 66 and soldered thereto. The distal window frame 66 surrounds in this case the ocular window 64 in the peripheral direction of the ocular window 64, wherein the ocular window 64 to the inner space of the surgical instrument 2 is in contact with a retaining shoulder 68 of the ocular window frame 62.

Similar to the window disk 20 shown in FIG. 2, the ocular window 64 is provided with a gold coating on the outer peripheral surface, and is additionally provided with an annular gold coating in the outer peripheral region on the flat level, or respectively flat surface that faces the inner space of the endoscope.

The window frame 66 is provided with a gold coating 24 on the inner sides that face the peripheral surface of the cylindrical ocular window 64. The contact surface of the retaining shoulder 68 that faces the annular coating of the ocular window 64 is also provided with a gold coating 24. A laser, such as a diode laser, is arranged on the ocular device 60 in order to heat the solder preform 40 (not shown here) while using a solder preform (not shown here) between the window frame 66 and the ocular window 64 in order to solder them to each other. By heating the solder preform, the gap between the ocular frame 66 and the ocular window 64 is filled, which solders the ocular window 64 in the ocular frame 66.

The ocular window frame 62 can be produced from steel or a plastic, such as PEEK.

While there has been shown and described what is considered to be preferred embodiments, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### LIST OF REFERENCE SIGNS

2 Surgical instrument

4 Endoscope shaft

6 Housing

8 Eyepiece

10 Light source

12 Connecting cable

14 Camera head

16 Connection

20 Window disk

22 Peripheral surface

24 Gold coating

26 Flat surface

30 Fiber tube

32 Ring body

36 Gold coating

40 Solder preform

50 Inductive heating device

60 Ocular device

62 Ocular window frame

64 Ocular window

66 Window frame

68 Shoulder

70 Laser device

1. A method for the manufacturing an endoscope, the method comprising:
providing a window having a lateral peripheral surface and a flat surface, wherein the window is provided with a metal coating on one or more of a lateral peripheral surface and an edge region of the flat surface, and wherein the flat surface of the window is provided with a solder preform,
inserting the window provided with the solder preform into a window seat of an endoscope shaft of the endoscope, wherein the flat surface of the window one of faces an interior space of the endoscope shaft or faces away from the interior space of the endoscope shaft, and
subsequent to the inserting, heating the solder preform with a heating device such that solder material of the solder preform is distributed between the window and the window seat and, after the solder material cools, solder is arranged is between the window and the window seat.

2. The method according to claim 1, wherein the heating comprises simultaneously heating an entirety of the solder preform.

3. The method according to claim 1, wherein the heating comprises heating less than an entirety of the solder preform at a predetermined number of positions.

4. The method according to claim 1, wherein each of the lateral peripheral surface and the flat surface include the metal coating.

5. The method according to claim 1, wherein the metal coating comprises gold.

6. The method according to claim 1, wherein the solder preform is configured to have one of an annular shape or a disk-shape.

7. The method according to claim 1, wherein the solder preform comprises gold and tin.

8. The method according to claim 1, wherein the heating comprises one of inductive heating or heating with a laser.

9. The method according to claim 1, wherein the window is configured in a cylindrical shape.

10. The method according to claim 1, wherein the window comprises sapphire glass.

11. The method according to claim 1, wherein the metal coating is formed on the edge portion of the flat surface of the window in a ring-shape.

12. The method according to claim 11, wherein the ring-shape has a ring width between 50 μm and 1000 μm.

13. A window for use in a window seat of an endoscope shaft of the endoscope, the window comprising:
a lateral peripheral surface;
a flat surface;
a solder preform provided to the flat surface; and
a metal coating on one or more of the lateral peripheral surface and an edge region of the flat surface.

14. The window according to claim 13, wherein the metal coating is comprises gold.

15. The window according to claim 13, wherein the solder preform is configured to have one of an annular shape or a disk-shape.

16. The window according to claim 13, wherein the solder preform comprises gold and tin.

17. The window according to claim 13, wherein the window is configured to have a cylindrical shape.

18. The window according to claim 13, wherein the window is formed from sapphire glass.

19. The window according to one claim 13, wherein the metal coating is formed on at least the flat surface in a ring shape.

20. The window according to claim 19, wherein the ring shape has a ring width between 50 μm and 1000 μm
